SEQUENCE LISTING (1 sequence)

SEQ ID NO: 1 - protein, 154 aa, Homo sapiens
MGKSASKQFHNEVLKAHNEYRQKHGVPPLKLCKNLNREAQQYSEALASTRILKHSPESSRGQCGENLAWASYDQTGKEVADRWYSEIKNYNFQQPGFTSGTGHFTAMVWKNTKKMGVGKASASDGSSFVVARYFPAGNVVNEGFFEENVLPPKK

(19) United States
(12) Patent Application Publication
(10) Pub. No.: US 2011/0059069 A1
(43) Pub. Date: Mar. 10, 2011

(54) Gapr-1 Methods

(75) Inventors: Ruth Baxter, Los Angeles, CA (US); Humphrey Gardner, Salem, MA (US)

(73) Assignee: Biogen Idec MA Inc

(21) Appl. No.: 11/992,464

(22) PCT Filed: Sep. 22, 2006
(86) PCT No.: PCT/US06/36972

§ 371 Date: Nov. 23, 2010

Related U.S. Application Data
(60) Provisional application No. 60/719,355, filed on Sep. 22, 2005.

(51) Int. Cl. A61K 39/395; C12N 5/077; G01N 33/53; G01N 33/00; A61K 38/16; C12N 5/071; A01N 1/02; A61P 35/00

(52) U.S. Cl. 424/133.1; 435/375; 424/130.1; 424/139.1; 436/86; 436/94; 435/7.21; 800/3; 514/19.3; 435/374; 435/1.1

(57) ABSTRACT

Methods of treatment, diagnosis and screening related to GAPR-1 are disclosed.

This non-provisional application claims benefit of priority of U.S. provisional application 60/719,355, filed Sep. 22, 2005. The entire contents of the aforementioned application are incorporated herein.

### BACKGROUND

Epithelial to mesenchimal transition (EMT) is a cellular process by which epithelial cells acquire phenotypic and functional characteristics of fibroblast-like cells. As a consequence of EMT, epithelial cells become elongated, mobile and lose their polarity and firm cellular junctions. EMT is a central mechanism for diversifying the cells found in complex tissues and is a primary mechanism for remodeling tissues during embryogenesis. In addition, EMT is involved in initiating metastasis of carcinoma cells, and in the genesis of fibroblasts in injured tissues. See, e.g., Kalluri and Neilson (2003) J. Clin. Invest. 112:1776-1784.

### SUMMARY OF THE INVENTION

The invention is based, at least in part, on the discovery that GAPR-1 is involved in epithelial to mesenchymal transition (EMT) and fibrosis. Accordingly, GAPR-1 is identified as a target for modulation of EMT and for the treatment of EMT-related conditions, such as fibrosis, kidney disease and cancer metastasis.

In one aspect, the invention features a method of modulating transition from epithelial to mesenchymal phenotype (EMT), and vice versa. The method includes contacting a cell, tissue or organ with an agent that modulates a GAPR-1 function, e.g., an agent that modulates GAPR-1 levels, expression or activity. The cell, tissue or organ can be from, e.g., kidney, lung, liver, skin, brain, prostate, pancreas, breast, prostate, colon, colorectal, ovary, cervix, brain, uterus, bladder, or testicle cell or tissue. The cell, tissue or organ can be, e.g., a fibrotic cell, tissue or organ, or a cell, tissue or organ from an epithelial tumor, e.g., a carcinoma or adenocarcinoma.

In one embodiment, the agent increases GAPR-1 level expression or activity to thereby increase EMT. In one embodiment, the agent is GAPR-1, e.g., a soluble GAPR-1, or a functional fragment thereof.

In another embodiment, the agent decreases GAPR-1 levels, expression or activity to thereby decrease EMT or promote mesenchymal to epithelial transition. Such an agent can be, e.g., a dominant-negative GAPR-1 protein, or an anti-GAPR-1 antibody (e.g., an inhibitory or blocking anti-GAPR-1 antibody) or antigen-binding fragment thereof. The antibody is typically a monospecific antibody, such as a monoclonal antibody, e.g., a humanized antibody, a chimeric antibody, or a fully human antibody. The antibody may bind an epitope of SEQ ID NO:1.

The method may be performed in vivo, ex vivo or in vitro.

In another aspect, the invention features a method of treating fibrosis in a subject, preferably a human. The method includes identifying a subject having or at risk for fibrosis, and administering to the subject an agent that reduces the amount of GAPR-1 levels, activity or expression. In one embodiment, the agent is administered in an amount and for a time sufficient to reduce one or more of: the amount of fibrotic tissue in the subject, the amount or rate of fibrogenesis in the subject, and the amount or rate of migration of epithelial cells into an interstitium.

In some embodiments, the subject has, or is at risk of, kidney disease and/or kidney fibrosis. In such embodiments, the agent can be administered in an amount and for a time sufficient to reduce one or more of: the amount of fibrotic tissue in the kidney, the amount or rate of fibrogenesis in the kidney, the amount or rate of migration of epithelial cells into an interstitium, and the time to or rate of progression to chronic renal disease in the subject.

In one embodiment, the agent inhibits GAPR-1 dimerization or multimerization. For example, the agent binds to one or more of: His54, Glu65, Glu86, and His103 of GAPR-1.

In one embodiment, the agent is a dominant negative GAPR-1 protein, e.g., the agent is a GAPR-1 protein in which one or more of His54, Glu65, Glu86, and His103 have been mutated.

In some embodiments, the agent is an inhibitory anti-GAPR-1 antibody (preferably a monospecific antibody such as a monoclonal antibody) or antigen-binding fragment thereof. In one embodiment, the agent is an antibody that is a full length IgG. In other embodiments, the agent is an antigen-binding fragment of a full length IgG, e.g., the agent is a single chain antibody, Fab fragment, F(ab′)2 fragment, Fd fragment, Fv fragment or dAb fragment. In some embodiments, the antibody is a human, humanized, chimeric or humaneered antibody or antigen-binding fragment thereof. In one embodiment, the antibody specifically binds an epitope within SEQ ID NO:1.

In some embodiments, the agent is a blocking anti-GAPR-1 aptamer.

In one embodiment, the subject has renal, pulmonary, skin or hepatic fibrosis.

In one embodiment, the method includes administering a second therapeutic agent for treating fibrosis, e.g., a TGF-beta pathway inhibitor, e.g., a TGF-beta pathway inhibitor described herein.

In one embodiment, the agent is administered at a dose between 0.1-100 mg/kg, between 0.1-10 mg/kg, between 1 mg/kg-100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In one embodiment, the method also includes evaluating the subject for a marker or diagnostic indication of fibrosis, e.g., using a CT scan or HRCS, biopsy or blood test for fibrosis. The evaluation can occur before and/or after the administration, e.g., to diagnose the subject and/or to monitor response to treatment. The evaluation can occur at least once, at least twice, 3, 4, 5, 6 or more times.

In another aspect, the invention features a method of evaluating a subject for risk of fibrosis or metastasis of a tumor. The method includes evaluating GAPR-1 protein or a nucleic acid encoding GAPR-1 in the subject or in a sample obtained from the subject. In one embodiment, increased GAPR-1 levels, activity or expression correlates with increased risk of fibrosis or metastasis of a tumor. “Correlating” means identifying the increased GAPR-1 levels, activity or expression as a risk or diagnostic factor for fibrosis or metastasis of a tumor, e.g., providing a print material or computer readable medium, e.g., an informational, diagnostic, marketing or instructional print material or computer readable medium, e.g., to the subject or to a health care provider, identifying the increased GAPR-1 levels, activity or expression as a risk or diagnostic factor for fibrosis or metastasis of a tumor.

In one embodiment, the methods include diagnosing a subject as being at risk for fibrosis or metastasis of a tumor. In one embodiment, the methods include prescribing or beginning a treatment for fibrosis or metastasis.

The subject is typically a human, e.g., a human with a family history of fibrosis or cancer (e.g., carcinoma).

The sample can be a cell sample, tissue sample, or at least partially isolated molecules, e.g., nucleic acids, e.g., genomic DNA, cDNA, mRNA, and/or proteins derived from the subject.

In one embodiment, the methods include contacting a biological sample, e.g., a blood or cheek cell sample, with a compound or an agent capable of detecting GAPR-1 protein or nucleic acid, such that the presence of GAPR-1 nucleic acid or protein is detected in the biological sample. In one embodiment, the compound or agent is a nucleic acid probe capable of hybridizing to GAPR-1 mRNA, or an antibody capable of binding to GAPR-1 protein. In some embodiments, the evaluation is used to choose a course of treatment.

In another aspect, the invention features methods of providing information, e.g., for making a decision with regard to the treatment of a subject having, or at risk for, a disorder described herein. The methods include (a) evaluating the expression, level or activity of GAPR-1; optionally (b) providing a value for the expression, level or activity of GAPR-1; optionally (c) comparing the provided value with a reference value, e.g., a control or non-disease state reference or a disease state reference; and optionally (d) based, e.g., on the relationship of the provided value to the reference value, supplying information, e.g., information for making a decision on or related to the treatment of the subject. In one embodiment, the decision is whether to administer a preselected treatment.

In another aspect, the invention features a method of identifying an agent that modulates EMT. The method includes identifying an agent that modulates the expression, activity or levels of GAPR-1, and correlating the ability of the identified agent to modulate the expression, activity or levels of GAPR-1 with the ability of the identified agent to modulate EMT. “Correlating” means identifying an agent that increases or decreases GAPR-1 levels, activity, or expression as an agent capable of modulating EMT (e.g., modulating fibrosis or metastasis). The correlating step can include, e.g., generating or providing a record (e.g., a print or computer readable record, such as a laboratory record or dataset or an email) identifying a test agent that decreases expression, activity or levels of GAPR-1 as an agent capable of promoting or increasing EMT. The record can include other information, such as a specific test agent identifier, a date, an operator of the method, or information about the source, structure, method of purification or biological activity of the test agent. The record or information derived from the record can be used, e.g., to identify the test agent as a compound or candidate agent (e.g., a lead compound) for pharmaceutical or therapeutic use. The identified agent can be identified as an agent or a potential agent for treatment of an EMT-related condition, e.g., an EMT-related condition described herein. Agents, e.g., compounds, identified by this method can be used, e.g., in the treatment (or development of treatments) for modulating EMT, fibrosis, metastasis, or kidney disease.

In one embodiment, the method includes providing a test agent and evaluating whether the test agent binds GAPR-1. In one embodiment, the method includes providing a test agent and evaluating whether the test agent affects the ability of GAPR-1 to affect EMT, e.g., in an assay described herein.

The “identifying” step can include (a) providing a cell, tissue (e.g., an epithelial cell or tissue) or non-human animal harboring an exogenous nucleic acid that includes a GAPR-1 regulatory region (e.g., a GAPR-1 promoter) operably linked to a nucleotide sequence encoding a reporter polypeptide (e.g., a light based, e.g., colorimetric or fluorescently detectable label, e.g., a fluorescent reporter polypeptide, e.g., Green Fluorescent Protein (GFP), Enhanced Green Fluorescent Protein (EGFP), Blue Fluorescent Protein (BFP), Red Fluorescent Protein (RFP)), (b) evaluating the ability of a test agent to modulate the activity of the reporter polypeptide in the cell, tissue or non-human animal and (c) selecting a test agent that increases or decreases the activity of the reporter polypeptide as an agent that modulates EMT. In some embodiments, the evaluation includes entering a value for the evaluation, e.g., a value for the effect of the test agent on GAPR-1, into a database or other record.

In one embodiment, the method includes two evaluating steps, e.g., the method includes a first step of evaluating the test agent in a first system, e.g., a cell-free, cell or tissue system, and a second step of evaluating the test agent in a second system, e.g., a second cell or tissue system or in a non-human animal. In other embodiments, the method includes two evaluating steps in the same type of system, e.g., the agent is re-evaluated in a non-human animal after a first evaluation in the same or a different non-human animal. The two evaluations can be separated by any length of time, e.g., days, weeks, months or years. In one embodiment, the test agent is first evaluated for its ability to interact with GAPR-1, e.g., bind to GAPR-1, and is then evaluated for its ability to modulate EMT, e.g., in vitro or in vivo.

The test agent can be a crude or semi-purified extract (e.g., an organic, e.g., animal or botanical extract) or an isolated compound, e.g., a small molecule, protein, lipid or nucleic acid.

In one embodiment the method includes evaluating the ability of the identified or selected agent to modulate EMT in vitro, ex vivo or in vivo.

In a further embodiment, the method includes evaluating the ability of the identified or selected agent to modulate fibrosis and/or metastasis in a non-human, experimental animal.

In another aspect, the invention features a method of treating a tumor, e.g., a cancer, in a subject, preferably a human. The method includes identifying a subject having or at risk for cancer, and administering to the subject an agent that reduces the levels, expression or activity of GAPR-1. In one embodiment, the agent is administered in an amount and for a time sufficient to reduce one or more of: the amount or rate of metastasis (e.g., bone metastasis, brain metastasis), invasiveness, the amount of cancer tissue in the subject (e.g., tumor volume), and the amount or rate of carcinogenesis in the subject.

In one embodiment the agent treats cancer in a subject by inhibiting GAPR-1 dimerization or multimerization. For example, the agent binds to one or more of: His54, Glu65, Glu86, and His103 of GAPR-1.

In one embodiment, the agent is a dominant negative GAPR-1 protein, e.g., the agent is a GAPR-1 protein in which one or more of His54, Glu65, Glu86, and His103 have been mutated.

In some embodiments, the agent is an inhibitory anti-GAPR-1 antibody (preferably a monospecific antibody such as a monoclonal antibody) or antigen-binding fragment thereof. In one embodiment, the agent is an antibody that is a full length IgG. In other embodiments, the agent is an antigen-binding fragment of a full length IgG, e.g., the agent is a single chain antibody, Fab fragment, F(ab′)2 fragment, Fd fragment, Fv fragment, or dAb fragment. In preferred embodiments, the antibody is a human, humanized, chimeric or humaneered antibody or antigen-binding fragment thereof In one embodiment, the antibody specifically binds an epitope within SEQ ID NO:1.

In some embodiments, the agent is an inhibitory anti-GAPR-1 aptamer.

In one embodiment, the subject has cancer, e.g. a carcinoma (such as an adenocarcinoma), including kidney, lung, liver, skin, brain, prostate, pancreas, breast, prostate, colon, colorectal, ovarian, cervix, brain, uterus, bladder, or testicular cancer.

In one embodiment, the method includes administering a second therapeutic agent for treating cancer, e.g., anti-angiogenic compounds, antiproliferative agents, anti-estrogens, anti-metabolites, kinase inhibitors, e.g., as described herein.

In one embodiment, the agent is administered at a dose between 0.1-100 mg/kg, between 0.1-10 mg/kg, between 1 mg/kg-100 mg/kg, between 0.5-20 mg/kg, or between 1-10 mg/kg. In the most typical embodiment, the dose is administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

In one embodiment, the method also includes evaluating the subject for a marker or diagnostic indication of cancer, e.g., using a CT scan or HRCS, biopsy or blood test for cancer.

In another aspect, the invention includes a method of maintaining a phenotype (e.g., mesenchymal or epithelial phenotype) of a cell by modulating GAPR-1.

In one embodiment, the method includes maintaining mesenchymal phenotype of a cell or tissue. The method includes contacting the cell or tissue in an in vitro or ex vivo culture with GAPR-1 or a functional fragment thereof. The method further can comprise growing or harvesting the culture. In an alternate embodiment the method of maintaining mesenchymal phenotype of a cell includes maintaining the mesenchymal phenotype of a stem or progenitor cell in culture. “Maintaining mesenchymal phenotype” means that one or more mesenchymal characteristic (e.g., up-regulation of vimentin, dispersion of cytokeratin, or loss of organized adhesion proteins at intercellular boundaries) is increased in the culture relative to the characteristic in the absence of the GAPR-1 or functional fragment thereof.

In one embodiment, the method includes maintaining the epithelial phenotype of cells or tissue in culture. The method includes contacting the cells or tissue in an in vitro or ex vivo culture with a GAPR-1 antagonist, e.g., a blocking anti-GAPR antibody or GAPR-1 dominant negative protein, whereby the epithelial phenotype is maintained. The method further can comprise growing or harvesting the culture. “Maintaining epithelial phenotype” means that one or more epithelial characteristics (e.g., epithelioid morphology; epithelial-type intercellular adhesion proteins localized to junctional complexes; keratin-containing intermediate filaments; and down-regulation of non-epithelial genes) is increased in the culture relative to the characteristic in the absence of the antagonist.

The term “treating” refers to administering a therapy in an amount, manner, and/or mode effective to improve or prevent a condition, symptom, or parameter associated with a disorder or to prevent onset, progression, or exacerbation of the disorder (including secondary damage caused by the disorder), to either a statistically significant degree or to a degree detectable to one skilled in the art. Accordingly, treating can achieve therapeutic and/or prophylactic benefits. An effective amount, manner, or mode can vary depending on the subject and may be tailored to the subject.

Each of the limitations of the invention can encompass various embodiments of the invention. It is, therefore anticipated that each of the limitations of the invention involving any one element or combinations of elements can be included in each aspect of the invention.

### BRIEF DESCRIPTION OF THE DRAWINGS

FIG. 1 is a Western blot with an anti-V5-tag antibody to detect V5-tagged C-terminal of GAPR-1. Lane 1 is a cell pellet 24 hours following transfection; lane 2 is conditioned medium 24 hours following transfection; lane 3 is MW markers.

FIG. 2A-C is a series of Western blots with polyclonal rabbit antiserum against the C-terminal portion of GAPR-1. W=conditioned medium from wild type, untransfected cells; T=conditioned medium from cells transfected with V5 tagged GAPR-1.

FIG. 3A-F is a series of immunostains of wild type and 7 week Alports kidneys with anti-GAPR-1.

FIG. 4A-D is an in vitro epithelial to mesenchymal transition assay. A: epithelial conditions; B: mesenchymal conditions; C: GAPR-1 conditioned medium; D: Antibody depleted GAPR-1 conditioned medium.

FIG. 5 is a Western blot for E-cadherin and vimentin expression as a function of depletion of GAPR-1 from conditioned medium.

### DETAILED DESCRIPTION OF THE INVENTION

The methods and compositions described herein relate to a role for GAPR-1 (Golgi-associated Pathogenesis Related Protein 1) in epithelial to mesenchymal transition (EMT), fibrosis, and cancer. GAPR-1 was found to be upregulated approximately 20× in fibrotic mouse kidneys. GAPR-1 caused epithelial cells to undergo transition from epithelial to mesenchymal phenotype, a model for the development of fibrosis and tumor metastasis. While not bound by theory, GAPR-1 inhibitors may work to inhibit fibrosis and cancer by reducing EMT.

GAPR-1 has also been identified as Golgi-associated Plant Pathogenesis Related Protein, C9or f19, and 17 kD fetal brain protein. (See, Eisenberg I, et. al. (2002) Gene 293:141-48; Eberle H B, et al. (2002) J. Cell Science 115: 827-38; Serrano et. al. (2004) J. Mol. Biol. 339: 173-83.)

### Fibrosis

Fibroblasts accumulate and promote scar formation as part of the body's natural response to tissue injury. This process can go awry due to a number of factors including trauma, chronic injury, inflammation, infection, and/or exposure to toxins, leading to an excessive production and deposition of collagen, and resulting in fibrosis or a fibrotic disorder.

Disorders that are primarily fibrotic include scleroderma, pulmonary fibrosis (e.g., idiopathic pulmonary fibrosis), liver fibrosis, renal fibrosis, and radiation induced fibrosis. Fibrosis can also occur as a symptom and/or result of various disorders or conditions, including atherosclerosis, nephropathy, hepatitis, restenosis, stroke, burns, wounds, and transplant rejection, pulmonary hypertension (PPH), broncopulmonary dysplasia (BPD), lung transplant rejection and pulmonary GVHD complications, interstitial pneumonia syndrome (IPS) in transplant recipients, acute lung injury (ALI)/acute respiratory distress (ARDS), COPD, HIV-associated nephropathy, IgA nephropathy, diabetic nephropathy, lupus nephritis, idiopathic glomerulosclerosis, kidney transplant rejection, renal complications of GVHD, autoimmune hepatitis, chronic viral hepatitis (Hepatitis B, Hepatitis C), primary sclerosing cholangitis (PSC), primary biliary cirrhosis (PBC), non-alcohol Steatohepatitis (NASH), liver transplant rejection, complications of GVHD, veno-occlusive disease in transplant recipients, acute wounds, chronic wounds, burns, surgical adhesions, keloids, donor-graft re-epithelialization, ocular scarring, restenosis, subarachnoid hermorrhage (SAH), heart transplant rejection, stroke, ophthalmic scarring, spinal cord injury, and cancer related fibrosis. An abnormal accumulation of fibrotic tissue can occur leading to the destruction of tissue and ultimately to inhibited organ function or failure.

Epithelial-mesenchymal transition (EMT) plays a role in the genesis of fibroblasts during organ fibrosis (Kalluri and Neilson (2003) J. Clin. Invest. 112(12): 1776-84).

### Cancer

Cancer is characterized by the uncontrolled, abnormal growth of cells.

Cancers of epithelial origin (carcinomas and adenocarcinomas) are often identified as a solid mass or tumor, but in many cases, can break apart and spread throughout the body as single cells, a process known as metastasis. One of the earliest events in the metastasis process is the loosening of the junctions with other cells in the primary tumor, followed by migration towards and invasion through the limiting structures these cells may encounter. The basement membrane is especially important in this regard because it surrounds the gland, as well as the blood vessels which the cancer cells need to penetrate to metastasize. Metastatic cancer cells can cross this membrane and invade other tissues. The ability of carcinoma cells to metastasize is believed to involve epithelial-to mesenchymal transition (EMT), which process results in loss of cell:cell adhesions, increased migration, and increased production of the enzymes capable of degrading the tissue barriers like the basement membrane. One indicator of EMT is the loss of keratin expression, and the gain of vimentin expression.

Carcinomas including adenocarcinoma can occur in any epithelial tissue, including pancreatic, breast, lung, prostate, colon, colorectal, skin, ovarian, cervical, brain, uterine, bladder, testicular or renal tissue.

### Organ Culture

During organ development cells arrange and rearrange themselves as they multiply, grow, and ultimately form the complex tissue layers of mammalian organs. An early example of this cell formation and subsequent rearrangement is formation of the three germ layers at the gastrulation phase.

To undergo this process the cells take and maintain their formation while in an epithelial phenotype. The epithelial phenotype includes cell-to-cell interactions and adhesions that give structure to tissue. When the tissue rearranges, the cells must break these cell-to-cell interactions by undergoing EMT. (Kerrigan, J J, et. al. (1998) J. R. Coll. Surg. Edinb., 43: 223-229.) The methods herein may be useful where it is desirable to maintain the mesenchymal phenotype, e.g., in cell culture.

### Inhibitors of GAPR-1 Function

Inhibitors of GAPR-1 are described herein as being useful in the treatment of fibrosis, cancer, kidney disease, and/or in the modulation of EMT. An inhibitor of GAPR-1 may be any type of compound (e.g., small organic or inorganic molecule, nucleic acid, protein, or peptide mimetic) that can be administered to a subject, e.g., blocking antibodies, dominant negative GAPR-1 polypeptides, small molecule antagonists, aptamers, and gene therapy technologies including RNAi and antisense compounds.

### Antibodies

The amino acid sequence of human GAPR-1 is shown below (SEQ ID NO:1)

| | |
|---|---|
| 1 | mgksaskqfh nevlkahney rqkhgvpplk lcknlnreaq qysealastr ilkhspessr |
| 61 | gqcgenlawa sydqtgkeva drwyseikny nfqqpgftsg tghftamvwk ntkkmgvgka |
| 121 | sasdgssfvv aryfpagnvv negffeenvl ppkk |

Naturally occurring GAPR-1 protein may be isolated from cells or tissues, or it may be produced recombinantly by a cell (e.g., a bacterial, yeast or mammalian cell such as a CHO cell) that carries an exogenous nucleic acid encoding the protein. In other embodiments, the recombinant polypeptide is produced by a process commonly known as gene activation, wherein a cell that carries an exogenous nucleic acid that includes a promoter or enhancer is operably linked to an endogenous nucleic acid that encodes the polypeptide. Such proteins, or fragments thereof, can be used, e.g., as immunogens to produce antibodies described herein.

Exemplary GAPR-1 blocking agents include antibodies that bind to GAPR-1. In on embodiment, the antibody inhibits the interaction between GAPR-1 and a GAPR-1 binding partner (e.g., between two GAPR-1 monomers). The antibody may physically block the interaction, decrease the affinity of GAPR-1 for its binding partner, disrupt or destabilize GAPR-1 complexes, sequester GAPR-1, or target GAPR-1 for degradation. In one embodiment, the antibody can bind to GAPR-1 at an epitope that includes one or more amino acid residues that participate in multimer formation, e.g., one or more of: His54, Glu65, Glu86, and His103 of GAPR-1. In another embodiment, the antibody can bind to residues that do not participate in the GAPR-1 binding. For example, the antibody can alter a conformation of GAPR-1 and thereby reduce binding affinity, or the antibody may sterically hinder GAPR-1 binding. In one embodiment, the antibody can prevent activation of a GAPR-1 mediated event or activity (e.g., EMT).

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two beta-sheets formed of about seven beta-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally occurring variable domain. For example, the sequence may omit one, two or more N- or C-terminal amino acids, internal amino acids, may include one or more insertions or additional terminal amino acids or may include other alterations. In one embodiment, a polypeptide that includes an immunoglobulin variable domain sequence can associate with another immunoglobulin variable domain sequence to form a target binding structure (or “antigen binding site”), e.g., a structure that interacts with GAPR-1 or a GAPR-1 receptor.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

### Antibody Generation

Antibodies that bind to GAPR-1 can be generated by a variety of means, including immunization, e.g., using an animal, or in vitro methods such as phage display. All or part of GAPR-1 can be used as an immunogen or as a target for selection. For example, GAPR-1, or a fragment thereof, can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; US 2003-0070185; U.S. Pat. No. 5,789,650; and WO 96/34096.

Non-human antibodies to GAPR-1 or a GAPR-1 receptor can also be produced, e.g., in a rodent. The non-human antibody can be humanized, e.g., as described in EP 239400; U.S. Pat. Nos. 6,602,503; 5,693,761; and 6,407,213, deimmunized, or otherwise modified to make it effectively human.

EP 239400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody. Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; and 5,530,101; Tempest et al. (1991) Biotechnology 9:266-271 and U.S. Pat. No. 6,407,213.

Fully human monoclonal antibodies that bind to GAPR-1 can be produced, e.g., using in vitro-primed human splenocytes, as described by Boerner et al. (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large nonimmunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol Today 2:371-8; and US 2003-0232333).

### Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

Antibodies, particularly full-length antibodies, e.g., IgGs, can be produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr-host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein-A or Protein-G.

Antibodies (and other proteins described herein) may also include modifications, e.g., modifications that alter Fc function, e.g., to decrease or remove interaction with an Fc receptor or with Clq, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fe domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody. The protein can be purified from the milk, or for some applications, used directly.

Methods described in the context of antibodies can be adapted to other proteins, e.g., GAPR-1 polypepitde variants described herein.

### Nucleic Acid Antagonists

In certain implementations, nucleic acid antagonists are used to decrease expression of an endogenous gene encoding GAPR-1. In one embodiment, the nucleic acid antagonist is a siRNA that targets mRNA encoding GAPR-1. Other types of antagonistic nucleic acids can also be used, e.g., a dsRNA, a ribozyme, a triple-helix former, or an antisense nucleic acid.

siRNAs are small double stranded RNAs (dsRNAs) that optionally include overhangs. For example, the duplex region of a siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947, U.S. 2003/0166282, 2003/0143204, 2004/0038278, and 2003/0224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides that hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding GAPR-1) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding GAPR-1. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N4—(C1-C12) alkylaminocytosines and N4,N4—(C1-C12) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N6—(C1-C12) alkylaminopurines and N6,N6—(C1-C12) dialkylaminopurines, including N6-methylaminoadenine and N6,N6-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C1-C30 alkyl, C2-C30 alkenyl, C2-C30 alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071;. 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

### Aptamers

Aptamers are macromolecules composed of nucleic acid, such as RNA or DNA, that bind tightly to a specific protein. Typically, aptamers are 15-60 nucleotides long. The chain of nucleotides forms intramolecular interactions that fold the molecule into a complex three-dimensional shape. The shape of the aptamer allows it to bind tightly against the surface of its target molecule.

Methods of making aptamers are as routine as those for making antibodies. The SELEX process is a method for the in vitro evolution of nucleic acid molecules with highly specific binding to target molecules and is described in U.S. Pat. No. 5,475,096, U.S. Pat. No. 5,270,163, and WO 91/19813. Aptamers have been made to bind over 100 target ligands and can contest antibodies in therapeutics, imaging, and diagnostics (Hicke and Stephens (2000) J. Clin. Invest. 106:923-8; Jayasena (1999) Clin. Chem. 45:1628-50).

For in vivo applications, aptamers can be modified to dramatically reduce their sensitivity to degradation by enzymes in the blood, e.g., they may be PEGylated, or modified nucleotides may be used in their production. The basic SELEX method has been modified to achieve a number of specific objectives. For example, U.S. Pat. No. 5,707,796 describes the use of the SELEX process in conjunction with gel electrophoresis to select nucleic acid molecules with specific structural characteristics, such as bent DNA. U.S. Pat. Nos. 5,763,177 and 6,011,577, describe a SELEX based method for selecting nucleic acid ligands containing photoreactive groups capable of binding and/or photocrosslinking to and/or photoinactivating a target molecule. U.S. Pat. No. 5,580,737 describes a method for identifying highly specific nucleic acid ligands able to discriminate between closely related molecules. U.S. Pat. No. 5,567,588, describes a SELEX-based method which achieves highly efficient partitioning between oligonucleotides having high and low affinity for a target molecule.

### Artificial Transcription Factors

Artificial transcription factors can also be used to regulate expression of GAPR-1. The artificial transcription factor can be designed or selected from a library, e.g., for ability to bind to a sequence in an endogenous gene encoding GAPR-1, e.g., in a regulatory region, e.g., the promoter. For example, the artificial transcription factor can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Natl. Acad. Sci. USA 92:344 for, among other things, methods for creating libraries of varied zinc finger domains.

Optionally, an artificial transcription factor can be fused to a transcriptional regulatory domain, e.g., an activation domain to activate transcription or a repression domain to repress transcription. In particular, repression domains can be used to decrease expression of endogenous genes encoding GAPR-1. The artificial transcription factor can itself be encoded by a heterologous nucleic acid that is delivered to a cell or the protein itself can be delivered to a cell (see, e.g., U.S. Pat. No. 6,534,261). The heterologous nucleic acid that includes a sequence encoding the artificial transcription factor can be operably linked to an inducible promoter, e.g., to enable fine control of the level of the artificial transcription factor in the cell.

### GAPR-1 Polypeptides

A GAPR-1 dominant negative polypeptide is useful in the compositions and methods described herein, e.g., to treat fibrosis, cancer (e.g., to reduce metastasis), or a kidney disease.

GAPR-1 is thought to exist as both a Monomeric unit and a dimeric unit both in vivo and solution. A highly conserved Ser71 from one GAPR-1 monomer is thought to interact with the highly conserved His54 on a second GAPR-1 to create a dimer with a catalytic triad. Alternatively, GAPR-1 may function as a dimer with a catalytic tetrad between His54, Glu65, Glu86, and His103. (See Serrano et. al. (2004) J. Mol. Biol. 339: 173-83. It is clear to the skilled artisan that functional variants (i.e., having the same functions) of SEQ ID NO:1 can be constructed by, for example, making substitutions of residues or sequences (e.g., making conservative substitutions) or deleting terminal or internal residues or sequences not needed for biological activity. A skilled artisan could, without undue experimentation, make conservative substitutions in SEQ ID NO:1 without affecting biological function. Likewise, a skilled artisan can make a non-conservative substitution in a critical residue (e.g., a highly conserved residue) to disrupt a GAPR-1 function, e.g., to produce a dominant negative GAPR-1 polypeptide, e.g., the amino acids necessary in dimer formation may be disrupted by substitutions of the amino acid residues which have different characteristics such as size, charge, or conformation that could prevent dimerization. In another example, cysteine residues can be deleted or replaced with other amino acids to prevent formation of unnecessary intramolecular disulfide bridges upon renaturation. Other approaches may involve amino acid modifications, for example, to enhance expression in a chosen expression system.

As used herein, a “GAPR-1 polypeptide” is a polypeptide that includes a full length GAPR-1 amino acid sequence (SEQ ID NO:1) or a functional fragment or domain thereof. A GAPR-1 polypeptide can also optionally include a heterologous (non-GAPR-1) amino acid sequence, e.g., a GAPR-1 fusions protein, wherein a soluble fragment of GAPR-1 is fused to a heterologous amino acid sequence such as a peptide tag, AP, or an Fc region of an Ig, e.g., of an IgG. A human GAPR-1 polypeptide is not limited to SEQ ID NO:1. A human GAPR-1 polypeptide can comprise a sequence that is at least 90%, preferably at least 95%, 96%, 98%, or 99% identical to SEQ ID NO:1, and has a functional activity of GAPR-1, e.g., it can affect EMT in an assay described herein. Also included is a GAPR-1 polypeptide that comprises SEQ ID NO:1 with up to 15 amino acid deletions, substitutions, or additions, and has a functional activity of GAPR-1, e.g., it can affect EMT in an assay described herein.

A “dominant negative” GAPR-1 polypeptide is a variant of GAPR-1 such that the variant inhibits a function of GAPR-1 in-vitro and/or in-vivo. Such variants can be generated by any number of methods, including random mutagenesis (e.g., PCR mutagenesis and saturation mutagenesis), directed mutagenesis (e.g., by introducing deletions, insertions, or substitutions of residues of the GAPR-1 sequence and testing them for function), alanine scanning mutagenesis, cassette mutagenesis (e.g., based on the technique described in Gene 34:315 (1985), and combinatorial mutagenesis. Dominant negative GAPR-1 polypeptides can be identified among such variants by assaying them for the desired function, e.g., for the ability to inhibit or reduce wildtype GAPR-1 function in an EMT assay as described herein, or for the ability to disrupt multimerization of GAPR-1 monomers.

Routine techniques for making recombinant polypeptides (e.g., recombinant GAPR-1 or fragments thereof) may be used to construct expression vectors encoding the polypeptides of interest using appropriate transcriptional/translational control signals and the protein coding sequences. (See, for example, Sambrook et al., Molecular Cloning: A Laboratory Manual, 3d Ed. (Cold Spring Harbor Laboratory 2001). These methods may include in vitro recombinant DNA and synthetic techniques and in vivo recombination, e.g., in vivo homologous recombination. Expression of a nucleic acid sequence encoding a polypeptide may be regulated by a second nucleic acid sequence that is operably linked to the polypeptide encoding sequence such that the polypeptide is expressed in a host transformed with the recombinant DNA molecule.

Expression vectors capable of being replicated in a bacterial or eukaryotic host comprising a nucleic acid encoding a polypeptide are used to transfect a host and thereby direct expression of such nucleic acid to produce the polypeptide, which may then be isolated. The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Routine techniques for transfecting cells with exogenous DNA sequences may be used in the present invention. Transfection methods may include chemical means, e.g., calcium phosphate, DEAE-dextran, or liposome; or physical means, e.g., microinjection or electroporation. The transfected cells are grown up by routine techniques. For examples, see Kuchler et al. (1977) Biochemical Methods in Cell Culture and Virology. The expression products are isolated from the cell medium in those systems where the protein is secreted from the host cell, or from the cell suspension after disruption of the host cell system by, e.g., routine mechanical, chemical, or enzymatic means.

These methods may also be carried out using cells that have been genetically modified by other procedures, including gene targeting and gene activation (see Treco et al. WO 95/31560, herein incorporated by reference; see also Selden et al. WO 93/09222).

Suitable host cells for expression of a polypeptide as described herein can be prokaryotic or eukaryotic. Preferred eukaryotic host cells include, but are not limited to, yeast and mammalian cells, e.g., Chinese hamster ovary (CHO) cells (ATCC Accession No. CCL61), NIH Swiss mouse embryo cells NIH-3T3 (ATCC Accession No. CRL1658), and baby hamster kidney cells (BHI). Other useful eukaryotic host cells include insect cells and plant cells. Exemplary prokaryotic host cells are E. coli and Streptomyces.

A polypeptide produced by a cultured cell as described herein can be recovered from the culture medium as a secreted polypeptide, or, if it is not secreted by the cells, it can be recovered from host cell lysates. As a first step in isolating the polypeptide, the culture medium or lysate is generally centrifuged to remove particulate cell debris. The polypeptide thereafter is isolated, and preferably purified, from contaminating soluble proteins and other cellular components, with the following procedures being exemplary of suitable purification procedures: fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; and gel filtration, e.g., with Sephadex™ columns (Amersham Biosciences). Protease inhibitors may be used to inhibit proteolytic degradation during purification. One skilled in the art will appreciate that purification methods suitable for the polypeptide of interest may require modification to account for changes in the character of the polypeptide upon expression in recombinant cell culture.

The purification of polypeptides may require the use of, e.g., affinity chromatography, conventional ion exchange chromatography, sizing chromatography, hydrophobic interaction chromatography, reverse phase chromatography, gel filtration or other conventional protein purification techniques. See, e.g., Deutscher, ed. (1990) “Guide to Protein Purification” in Methods in Enzymology, Vol. 182.

### Gene Therapy

An agent described herein, such as an anti-GAPR-1 antibody or dominant negative GAPR-1 polypeptide, can be produced in vivo in a mammal, e.g., a human patient, using a gene therapy approach to treatment of fibrosis, cancer, or other condition in which reducing or reversing EMT would be therapeutically beneficial. This typically involves administration of a suitable an anti-GAPR-1 antibody or dominant negative GAPR-1 polypeptide-encoding nucleic acid operably linked to suitable expression control sequences. Preferably, these sequences are incorporated into a vector, e.g., a viral vector.

Expression constructs of an anti-GAPR-1 antibody or dominant negative GAPR-1 polypeptide may be administered in any biologically effective carrier, e.g. any formulation or composition capable of effectively delivering an anti-GAPR-1 antibody or dominant negative GAPR-1 gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (e.g., lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA, encoding a an anti-GAPR-1 antibody or dominant negative GAPR-1 polypeptide, or a GAPR-1 antisense nucleic acid. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes. A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989, with supplements, 2006), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include .psi.Crip, .psi.Cre, .psi.2 and .psi.Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Reviewed in Ali, 2004, Novartis Found Symp. 255:165-78; and Lu, (2004), Stem Cells Dev. 13(1):133-45. Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of an anti-GAPR-1 antibody or dominant negative GAPR-1 polypeptide, GAPR-1 fragment, or analog, in the tissue of an animal. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of an anti-GAPR-1 antibody or dominant negative GAPR-1 polypeptide gene by the targeted cell. Exemplary gene delivery systems of this type include liposomal derived systems, poly-lysine conjugates, dendrimers and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding an anti-GAPR-1 antibody or dominant negative GAPR-1 polypeptide, active fragment, or analog, can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). In clinical settings, the gene delivery systems for the therapeutic an anti-GAPR-1 antibody or dominant negative GAPR-1 gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

### Formulations

Compositions containing an agent described herein, e.g., GAPR-1 polypeptides, anti-GAPR-1 antibodies, or other therapeutic agents, may contain suitable pharmaceutically acceptable carriers. For example, they may contain excipients and/or auxiliaries that facilitate processing of the active compounds into preparations designed for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension include, for example, sodium carboxymethyl cellulose, sorbitol and dextran. Optionally, the suspension may also contain stabilizers. Liposomes also can be used to encapsulate the molecules of the invention for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In some embodiments, the compositions comprise pharmaceutically acceptable substances such as wetting or minor amounts of auxiliary substances such as wetting or emulsifying agents, preservatives or buffers, which enhance the shelf life or effectiveness of the active ingredients.

Compositions of the invention may be in a variety of forms, including, for example, liquid (e.g., injectable and infusible solutions), dispersions, suspensions, semi-solid and solid dosage forms. The preferred form depends on the mode of administration and therapeutic application. The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems! Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In some embodiments, a GAPR-1 polypeptide, anti-GAPR-1 antibody or fragment thereof is coadministered with at least one other therapeutic agent useful in treating fibrosis.

Dosage regimens may be adjusted to provide the optimum desired response. For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form may contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

### Administration

The GAPR-1 blocking agent (e.g., an antibody) can be administered to a subject, e.g., a human subject, by a variety of methods. For many applications, the route of administration is one of: intravenous injection or infusion (IV), subcutaneous injection (SC), intraperitoneally (IP), or intramuscular injection. In some cases, administration may be directly into the CNS, e.g., intrathecal or intracerebroventricular (ICV). The blocking agent can be administered as a fixed dose, or in a mg/kg dose.

The dose can also be chosen to reduce or avoid production of antibodies against the GAPR-1 blocking agent.

The route and/or mode of administration of the blocking agent can also be tailored for the individual case, e.g., by monitoring the subject, e.g., using assessment criteria discussed herein.

Dosage regimens are adjusted to provide the desired response, e.g., a therapeutic response. For example, doses in the range of 0.1-100 mg/kg, 1 mg/kg-100 mg/kg, 0.5-20 mg/kg, 0.1-10 mg/kg or 1-10 mg/kg can be administered. A particular dose may be administered more than once, e.g., at periodic intervals over a period of time (a course of treatment). For example, the dose may be administered every 2 months, every 6 weeks, monthly, biweekly, weekly, or daily, as appropriate, over a period of time to encompass at least 2 doses, 3 doses, 5 doses, 10 doses, or more.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

Alternatively, or in addition, the blocking agent may be administered via continuous infusion. The treatment can continue for days, weeks, months or even years.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic or detrimental effects of the composition are outweighed by the therapeutically beneficial effects.

### Combination Therapies

The methods and compositions described herein can be used in combination with other therapies for fibrosis, cancer or kidney disease, such as another biologic therapeutic, or a chemotherapeutic agent.

For example, a GAPR-1 inhibitor can be used with compounds useful in mitigating the effects of fibrosis such as steroid, e.g. a corticosteriod, e.g. prednisone, or a drug to suppress the body's immune response, e.g. azathioprine or cyclophosphamide, or inhibitors of collagen synthesis, e.g. pirfenidone. Still other compositions that can be used with a GAPR-1 inhibitor to treat fibrosis include compositions useful in regulating TGF-beta, including antibodies or aptamers against TGF-beta, i.e. TGF-beta1, TGF-beta2, TGF-beta3, antibodies or aptamers against TGF-beta receptors, i.e. TGF-beta RI, TGF-beta RII, soluble TGF-beta RI, soluble TGF-beta RII, antibodies or aptamers against compounds that are responsible for activating TGF-beta, i.e. alphav/beta6, compositions that regulate transcription factors in the TGF-beta pathway, and compositions that regulate TGF-beta signaling, i.e. endoglin.

Other compositions that can be used with a GAPR-1 inhibitor include compounds that are useful for reducing blood pressure, hyperlipidemia and hyperglycaemia.

Other compositions that can be used with a GAPR-1 inhibitor include compositions useful in treating cancer, i.e. carcinoma or adenocarcinoma, including anti-angiogenic compounds, e.g., anti-VEGF antibodies such as Avastin (bevacizumab); tyrosine kinase inhibitors; antiproliferative agents, e.g., an alkylating agent (e.g., dacarbazine), an anthracycline (e.g., mitoxantrone), an anti-estrogen (e.g., bicalutamide), an anti-metabolite (e.g., floxuridine), a microtubule binding, stabilizing agent (e.g., docetaxel), microtubule binding, destabilizing agent (e.g., vinorelbine), topoisomerase inhibitor (e.g., hydroxycamptothecin (SN-38)), or a kinase inhibitor (e.g., a tyrphostin, such as AG1478). The agent can be altretamine, carmustine, chlorambucil, cyclophosphamide, dacarbazine, ifosfamide, melphalan, mitomycin, temozolomide, doxorubicin, epirubicin, mitoxantrone, anastrazole, bicalutamide, estramustine, exemestane, flutamide, fulvestrant, tamoxifen, toremifene, capecitabine, floxuridine, fluorouracil, gemcitabine, hydroxyurea, methotrexate, gleevec, tyrphostin, docetaxel, pacilitaxel, vinblastine, vinorelbine, adjuvant/enhancing agents (celecoxib, gallium, isotretinoin, leucovorin, levamisole, pamidronate, suramin), or agents such as thalidomide, carboplatin, cisplatin, oxaliplatin, etoposide, hydroxycamptothecin, irinotecan, or topotecan. Other agents include antiproliferative agents selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, or rituximab

### Nucleic Acid and Protein Analysis

For evaluating a subject, e.g., in a diagnostic method, numerous methods for detecting GAPR-1 protein and nucleic acid are available, including antibody-based methods for protein detection (e.g., Western blot or ELISA), and hybridization-based methods for nucleic acid detection (e.g., PCR or Northern blot). Electrophoretic techniques include capillary electrophoresis and Single-Strand Conformation Polymorphism (SSCP) detection (see, e.g., Myers et al. (1985) Nature 313:495-8 and Ganguly (2002) Hum Mutat. 19(4):334-42). Other biophysical methods include denaturing high pressure liquid chromatography (DHPLC). Enzymatic methods for detecting nucleotide sequences include amplification based-methods such as the polymerase chain reaction (PCR; Saiki, et al. (1985) Science 230:1350-1354) and ligase chain reaction (LCR; Wu. et al. (1989) Genomics 4:560-569; Barringer et al. (1990), Gene 1989:117-122; F. Barany (1991) Proc. Natl. Acad. Sci. USA 1988:189-193); transcription-based methods utilize RNA synthesis by RNA polymerases to amplify nucleic acid (U.S. Pat. Nos. 6,066,457; 6,132,997; and 5,716,785; Sarkar et al., (1989) Science 244:331-34; Stofler et al., (1988) Science 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tm, e.g., between 45-60° C., 55-65° C., or 60-75° C. In a multiplex assay, Tms can be selected to be within 5, 3, or 2° C. of each other.

It is also possible to directly sequence the nucleic acid for a particular genetic locus, e.g., by amplification and sequencing, or amplification, cloning and sequence. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

Any combination of the above methods can also be used. The above methods can be used to evaluate any genetic locus, e.g., in a method for analyzing genetic information from particular groups of individuals or in a method for analyzing a polymorphism associated with fibrosis, e.g., in a gene encoding GAPR-1 or GAPR-1-R.

### Arrays

Arrays are particularly useful molecular tools for characterizing a sample, e.g., a sample from a subject. For example, an array having capture probes for multiple genes, including probes for GAPR-1 or for multiple proteins, can be used in a method described herein. Arrays can have many addresses, e.g., locatable sites, on a substrate. The featured arrays can be configured in a variety of formats, non-limiting examples of which are described below.

The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. Arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145).

The capture probe can be a single-stranded nucleic acid, a double-stranded nucleic acid (e.g., which is denatured prior to or during hybridization), or a nucleic acid having a single-stranded region and a double-stranded region. In one embodiment, the capture probe is single-stranded. The capture probe can be selected by a variety of criteria, and may be designed by a computer program with optimization parameters. The capture probe can be selected to hybridize to a sequence rich (e.g., non-homopolymeric) region of the gene. The Tm of the capture probe can be optimized by prudent selection of the complementarity region and length. Ideally, the Tm of all capture probes on the array is similar, e.g., within 20, 10, 5, 3, or 2° C. of one another.

The isolated nucleic acid can be mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y, 1989 with supplements, 2006). The substrate is washed, and the mRNA is eluted.

The isolated mRNA can be reversed transcribed and optionally amplified, e.g., by rtPCR, e.g., as described in (U.S. Pat. No. 4,683,202). The nucleic acid can be an amplification product, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

The expression level of a GAPR-1 protein can be determined using an antibody specific for the polypeptide (e.g., using a western blot or an ELISA assay). Moreover, the expression levels of multiple proteins, including GAPR-1 and at least one other fibrosis marker can be rapidly determined in parallel using a polypeptide array having antibody capture probes for each of the polypeptides. Antibodies specific for a polypeptide can be generated by a method described herein (see “Antibody Generation”).

A low-density (96 well format) protein array has been developed in which proteins are spotted onto a nitrocellulose membrane (Ge (2000) Nucleic Acids Res. 28, e3, I-VII). A high-density protein array (100,000 samples within 222×222 mm) used for antibody screening was formed by spotting proteins onto polyvinylidene difluoride (PVDF) (Lueking et al. (1999) Anal. Biochem. 270:103-111). See also, e.g., Mendoza et al. (1999). Biotechniques 27:778-788; MacBeath and Schreiber (2000) Science 289:1760-1763; and De Wildt et al. (2000). Nature Biotech. 18:989-994. These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

The nucleic acid and polypeptide arrays of the invention can be used in wide variety of applications. For example, the arrays can be used to analyze a patient sample. The sample is compared to data obtained previously, e.g., known clinical specimens or other patient samples. Further, the arrays can be used to characterize a cell culture sample, e.g., to determine a cellular state after varying a parameter, e.g., exposing the cell culture to an antigen, a transgene, or a test compound.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date and other quality control information.

Expression profiles obtained from gene expression analysis on an array can be used to compare samples and/or cells in a variety of states as described in Golub et al. ((1999) Science 286:531). In one embodiment, expression (e.g., mRNA expression or protein expression) information for a gene encoding GAPR-1 is evaluated, e.g., by comparison to a reference value. Reference values can be obtained from a control or a reference subject. Reference values can also be obtained from statistical analysis, e.g., to provide a reference value for a cohort of subjects, e.g., age and gender matched subjects, e.g., normal subjects or subjects who have fibrosis. Statistical similarity to a particular reference (e.g., to a reference for a risk-associated cohort) or a normal cohort can be used to provide an assessment (e.g., an indication of fibrosis risk) to a subject.

Subjects suitable for treatment can also be evaluated for expression and/or activity of GAPR-1. Subjects can be identified as suitable for treatment if the expression and/or activity for GAPR-1 is elevated relative to a reference, e.g., reference value, e.g., a reference value associated with normal.

Subjects who are being administered an agent described herein or other fibrosis treatment can be evaluated as described for expression and/or activity of GAPR-1. The subject can be evaluated at multiple times. e.g., at multiple times during a course of therapy, e.g., during a therapeutic regimen. Treatment of the subject can be modified depending on how the subject is responding to the therapy. For example, a reduction in GAPR-1 expression or activity can be indicative of responsiveness.

Particular effects mediated by an agent may show a difference (e.g., relative to an untreated subject, control subject, or other reference) that is statistically significant (e.g., P value<0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02.

### In Vivo Imaging

GAPR-1 blocking agents (e.g., antibodies) provide a method for detecting the presence of GAPR-1 in vivo (e.g., in vivo imaging in a subject), respectively. The method can be used to evaluate (e.g., diagnose, localize, or stage) a condition described herein, e.g., fibrosis, kidney disease or risk of fibrosis or kidney disease. The method includes: (i) administering to a subject (and optionally a control subject) a GAPR-1 binding agent (e.g., a blocking agent that binds to GAPR-1, e.g., an antibody or antigen binding fragment thereof), under conditions that allow interaction of the binding agent and GAPR-1 to occur; and (ii) detecting formation of a complex between the binding agent and GAPR-1, wherein detection of a complex identifies GAPR-1 expressing cells. A statistically significant increase in the amount of the complex in the subject relative to the reference, e.g., the control subject or subject's baseline, can be a factor that may lead to a diagnosis of fibrosis or risk for fibrosis.

Preferably, the GAPR-1 binding agent used in the in vivo (and also in vitro) diagnostic methods is directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the GAPR-1 binding protein is coupled to a radioactive ion, e.g., indium (¹¹¹In), iodine (¹³¹I or ¹²⁵I), yttrium (⁹⁰Y) actinium (²²⁵Ac), bismuth (²¹²Bi or ²¹³Bi), sulfur (³⁵S), carbon (¹⁴C), tritium (³H), rhodium (¹⁸⁸Rh) or phosphorous (³²P). In another embodiment, the GAPR-1 binding protein is labeled with an NMR contrast agent. In one aspect, the invention features a method of imaging vasculature in a patient who is at risk for fibrosis, has experienced fibrosis, and/or is recovering from fibrosis. The method includes: providing an agent that binds to GAPR-1 or GAPR-1-R, e.g., an agent described herein, wherein the protein is physically associated to an imaging agent; administering the agent to a patient, e.g., with a risk for fibrosis; and imaging the patient, e.g., to detect GAPR-1 or GAPR-1-R expressing cells.

### Methods of Screening

In another aspect, the invention features a method of screening for an agent that modulates, e.g., increases or decreases, EMT, and/or treats fibrosis or fibrotic transition. The method includes identifying an agent that reduces the expression, activity and/or levels of GAPR-1. The method can also include correlating the effect of the test agent on GAPR-1 with the test agent's ability to inhibit or decrease EMT (e.g., providing print material or a computer readable medium, e.g., informational, marketing or instructional print material or computer readable medium, related to the identified agent or its use). Correlating means identifying a test agent that decreases expression, activity or levels of GAPR-1 as an agent capable of inhibiting or decreasing EMT and/or reducing fibrosis).

The test agent can be a crude or semi-purified extract (e.g., an organic, e.g., animal or botanical extract) or an isolated compound, e.g., a small molecule, protein, lipid or nucleic acid. The test compounds of the screening assays described herein can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, to identify compounds that interfere with the interaction between two GAPR-1 monomers, a reaction mixture containing GAPR-1 is prepared, under conditions and for a time sufficient, to allow the two monomers to form a complex. To test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

All cited patents and publications are incorporated herein by reference.

### Examples

The invention is further illustrated by the following experimental examples. The examples are provided for illustrative purposes only, and are not to be construed as limiting the scope or content of the invention in any way.

### Example 1

### Cloning and Characterization of GAPR-1

GAPR-1 was cloned from a human kidney cDNA library. The cloned GAPR-1 was inserted into a Gateway expression vector (Invitrogen Gateway® Technology) containing a C-terminal V5 epitope and 6-His tags, under the control of either a CMV or an EF-1a promoter.

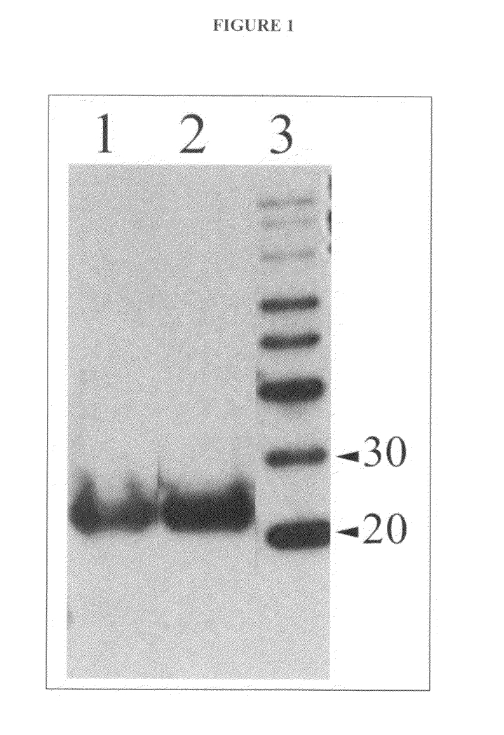

The expression construct was transfected into 293F cells (Invitrogen) and protein expression monitored by western blotting for the V5tag in cell pellets and conditioned medium collected after 24 hours (FIG. 1). The presence of GAPR-1 in the conditioned medium indicates that it is a secreted protein.

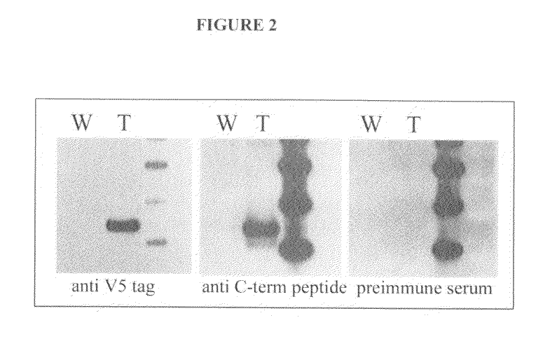

Peptides were designed to the carboxy terminus of the human GAPR-1 protein, and used to immunize rabbits to produce a polyclonal antibody to the GAPR-1 protein itself. This antibody recognized the same protein in conditioned media as the antibody against the V5 epitope tag (FIG. 2), confirming that GAPR-1 is soluble and secreted.

### Example 2

### GAPR1 is Increased in Fibrotic Kidney

Fibrotic mouse kidneys from a 7-week mouse were immunostained using the polyclonal serum described in Example 1. Prominent staining is present in the proximal tubule epithelium, which is blocked by the immunizing peptide, indicating that it is specific for GAPR-1 (not shown).

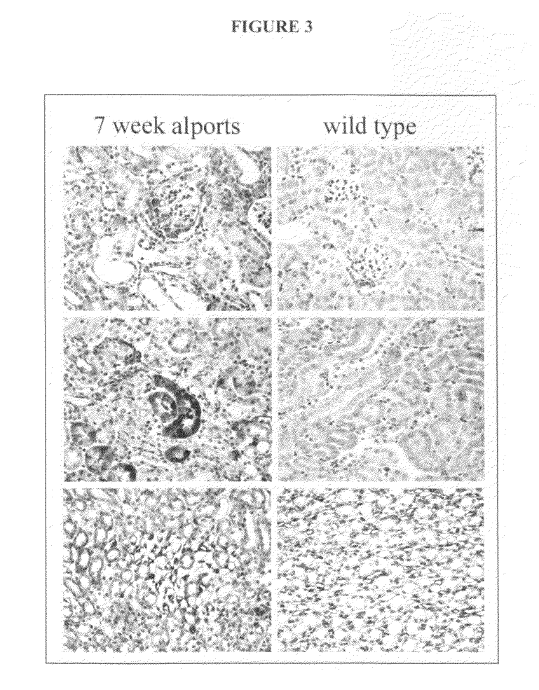

To determine if GAPR-1 expression is increased in fibrotic tissue, healthy wild type kidneys and fibrotic kidneys from 7 wk Alports mice (a model for progressive microscopic haematuria leading to chronic renal failure, see Cosgrove et al. (2000) Am J Pathol 157(5): 1649-59) were stained for the presence of GAPR-1 with the polylconal serum (FIG. 3). Examination of GAPR-1 staining in fibrotic kidney indicated high levels of expression in the damaged glomerulus, both in the cells of Bowman's capsule, and in the glomerulus itself. No GAPR-1 staining was observed in the glomerulus of a healthy wild type kidney, nor was significant staining seen in healthy glomeruli in the fibrotic kidneys. Prominent expression of GAPR-1 was observed in proximal tubule epithelium of fibrotic kidney, none is seen in wild type. GAPR-1 expression is also present in the collecting ducts of wild type kidneys, with a significant increase in expression level in fibrotic samples (FIG. 3).

In the damaged glomerulus in 7 week Alports kidneys, expression of GAPR-1 and smooth muscle actin are coincident (not shown). Expression of GAPR-1 in the proximal tubules is in the epithelial cells, and is not coincident with expression of smooth muscle actin, suggesting that expression of GAPR-1 may be an early step in the fibrotic process. In the collecting ducts, increased GAPR-1 staining is observed in the fibrotic kidney.

### Example 3

### GAPR-1 Induces EMT

This example shows that GAPR-1 promotes the differentiation of epithelial tissue toward the mesenchymal phenotype.

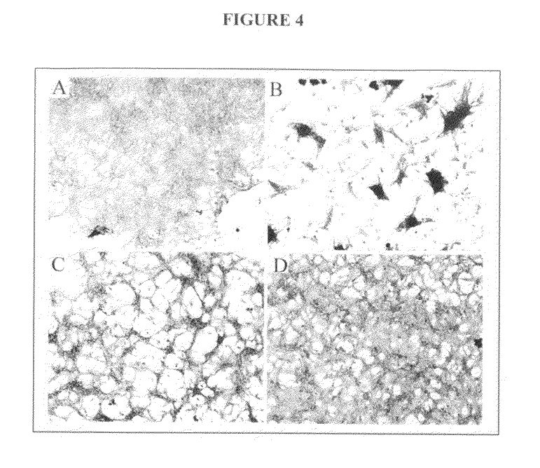

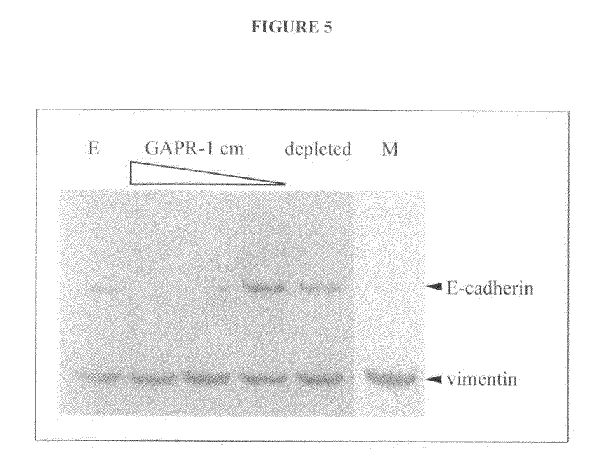

FIG. 4 shows the results of an EMT assay. Mouse kidney proximal tubule epithelial cells were cultured in the presence of TGFβ and EGF and induced to differentiate into mesenchymal cells (FIG. 4B), while those cultured in minimal medium grew into an epithelial monolayer (FIG. 4A). Addition of conditioned medium containing GAPR-1 to the minimal medium caused differentiation toward the mesenchymal phenotype in the absence of-added TGFβ or EGF (FIG. 4C). Prior incubation of the conditioned medium with C-terminal anti GAPR-1 to deplete the protein inhibited the differentiation caused by adding untreated conditioned medium containing GAPR-1 protein. (FIG. 4 compare panels C and D), indicating that the effect is GAPR-1 specific. (See Zeisberg et al. (2001) American Journal of Pathology 159(4): 1313-21 for a description of the EMT assay) A loss of expression of E-cadherin (a marker of epithelial cells) and an increase in expression of vimentin (a marker of mesenchymal cells) are markers of EMT. The GAPR-1 effect on EMT is coincident with these molecular markers. As shown in FIG. 5, in the presence of GAPR-1 conditioned medium the epithelial cells lose expression of E-cadherin. Depletion of GAPR-1 from the conditioned medium (cm) using the polyclonal serum results in no decrease of E-cadherin expression, indicating that this is a GAPR-1 specific effect.

1. A method of modulating epithelial-mesenchymal transition (EMT), in a cell or tissue, the method comprising contacting the tissue with an agent that modulates the level, expression, or activity of GAPR-1 in the tissue, thereby modulating EMT.

2. The method of claim 1, wherein the cell or tissue is a renal, pulmonary, hepatic, skin, pancreatic, breast, prostate, colon, colorectal, ovarian, cervical, brain, uterine, bladder, or testicular cell or tissue.

3. The method of claim 1, wherein the agent increases GAPR-1 level expression or activity to thereby increase EMT.

4. The method of claim 1, wherein the agent decreases GAPR-1 level expression or activity to thereby decrease EMT.

5. The method of claim 1, wherein the tissue is a fibrotic tissue.

6. The method of claim 1, wherein the tissue is a solid tumor.

7. The method of claim 6, wherein the tumor is a carcinoma.

8. The method of claim 3, where in the agent is GAPR-1.

9. The method of claim 4, wherein the agent is a dominant-negative GAPR-1 protein that decreases GAPR-1 levels, expression, or activity.

10. The method of claim 4, wherein the agent is an inhibitory anti-GAPR-1 antibody or antigen-binding fragment thereof.

11. The method of claim 10, wherein the antibody is a monoclonal antibody (mAb).

12. The method of claim 11, wherein the mAb is selected from the group consisting of: a humanized antibody, a chimeric antibody, and a human antibody.

13. The method of claim 10, wherein the antibody specifically binds an epitope within SEQ ID NO:1.

14. A method of treating fibrosis in a subject, the method comprising identifying a subject with fibrosis, and administering to the subject an agent that reduces the amount of GAPR-1 in a fibrotic tissue of the subject.

15. The method of claim 14, wherein the subject is a human.

16. The method of claim 14, wherein the agent reduces the amount of GAPR-1 by reducing GAPR-1 protein levels or activity.

17. The method of claim 16, wherein the agent inhibits GAPR-1 dimerization.

18. The method of claim 16, wherein the agent binds to an epitope comprising one or more of: His54, Glu65, Glu86, and His103 of GAPR-1.

19. The method of claim 16 wherein the agent is a dominant negative GAPR-1 protein.

20. The method of claim 16, wherein the agent is an inhibitory anti-GAPR-1 antibody or antigen-binding fragment thereof.

21. The method of claim 20, wherein the antibody is a mAb.

22. The method of claim 21, wherein the mAb is selected from the group consisting of: a humanized antibody, a chimeric antibody, and a human antibody.

23. The method of claim 20, wherein the mAb specifically binds an epitope within SEQ ID NO:1.

24. The method of claim 14, wherein the subject has renal, pulmonary, hepatic, cardiovascular, skin, ocular, nervous, or muscle fibrosis.

25. The method of claim 14, further comprising administering a second therapeutic agent for treating fibrosis.

26. A method of evaluating a subject for risk of fibrosis or metastasis of a tumor, the method comprising evaluating GAPR-1 protein or a nucleic acid encoding GAPR-1 in the subject or in a sample obtained from the subject, wherein increased GAPR-1 levels, activity or expression correlates with increased risk of fibrosis or metastasis of a tumor.

27. The method of claim 26, wherein GAPR-1 mRNA or DNA are evaluated.

28. The method of claim 26, wherein protein levels are quantitated.

29. The method of claim 26, further comprising providing a diagnosis or an assessment of fibrosis or metastasis as a function of the result of the evaluating.

30. A method of identifying an agent that modulates EMT, the method comprising:
identifying an agent that modulates the expression, activity or levels of GAPR-1, and correlating the ability of the identified agent to modulate the expression, activity or levels of GAPR-1 with the ability of the identified agent to modulate EMT.

31. The method of claim 30, wherein the identifying step comprises:
(a) providing a cell, tissue or non-human animal harboring an exogenous nucleic acid that includes a GAPR-1 promoter operably linked to a nucleotide sequence encoding a reporter polypeptide,
(b) evaluating the ability of a test agent to modulate the activity of the reporter polypeptide in the cell, tissue or non-human animal; and
(c) electing a test agent that increases or decreases the attivity of the reporter polypeptide as an agent that modulates EMT.

32. The method of claim 30, wherein the method further comprises evaluating the ability of the identified or selected agent to modulate EMT in vitro, ex vivo or in vivo.

33. The method of claim 30, wherein the method further comprises evaluating the ability of the identified or selected agent to modulate fibrosis and/or metastasis in a non-human, experimental animal.

34. A method of treating cancer, the method comprising identifying a subject with cancer, and administering to the subject an agent that reduces the amount, expression or activity of GAPR-1.

35. The method of claim 34, wherein the subject is a human.

36. The method of claim 34, wherein the agent inhibits GAPR-1 dimerization.

37. The method of claim 34, wherein the agent binds to one or more of: His54, Glu65, Glu86 and His103 of GAPR-1.

38. The method of claim 34, wherein the agent is a dominant negative GAPR-1 protein.

39. The method of claim 34, wherein the agent is an inhibitory anti-GAPR-1 antibody or antigen-binding fragment thereof.

40. The method of claim 39, wherein the antibody is a mAb.

41. The method of claim 40, wherein the mAb is selected from the group consisting of: a humanized antibody, a chimeric antibody, and a human antibody.

42. The method of claim 40, wherein the mAb specifically binds an epitope within SEQ ID NO:1.

43. The method of claim 34, wherein the cancer is a carcinoma.

44. The method of claim 34 further comprising administering a second therapeutic agent for treating cancer.

45. A method of maintaining mesenchymal phenotype of a cell or tissue, the method comprising contacting the cell or tissue with GAPR-1 or a functional fragment thereof.

46. The method of claim 45, wherein the cell or tissue is in an organ culture.

47. The method of claim 45, wherein the contacting occurs in vitro or ex vivo.

48. The method of claim 46, further comprising growing or harvesting the organ culture.

49. The method of claim 45, wherein the cell is, or the tissue comprises a stem cell or progenitor cell.
